(19) United States
(12) Patent Application Publication
(10) Pub. No.: US 2023/0102045 A1
(43) Pub. Date: Mar. 30, 2023

(54) CHEMICALS AND USE OF HYPOHALITES IN MECHANISM-BASED SELECTIVE DUAL RADICAL ORGANIC SYNTHESES

(71) Applicant: WALKER CANCER RESEARCH INSTITUTE, INC, Boynton Beach, FL (US)

(72) Inventor: Eduardo Palomino, Troy, MI (US)

(73) Assignee: WALKER CANCER RESEARCH INSTITUTE, INC, Boynton Beach, FL (US)

(21) Appl. No.: 17/394,680

(22) Filed: Aug. 5, 2021

(51) Int. Cl. C07B 35/04; C07B 37/06; C07B 41/02; C07B 37/10

(52) U.S. Cl. CPC C07B 35/04; C07B 37/06; C07B 41/02; C07B 37/10; C07D 209/42

(57) ABSTRACT

Embodiments of the present invention provide for syntheses of pattern-specific compounds using hypohalites, such as hypochlorous acid, sodium hypochlorite and potassium hypoiodite, as dual-radical generators, wherein the synthesis can be implemented by a cyclization reaction, a dehydrogenation reaction, a hydroxylation reaction, a decarboxylation reaction, or any combination of the above four.

### FIELD OF DISCLOSURE

The present invention relates the use of hypohalites in mechanism-based selective dual-radical organic syntheses.

### BACKGROUND OF THE INVENTION

The use of radicals in organic syntheses is abundant in the industry and employs mostly toxic materials that generate harmful by-products

Accordingly, there is a need for safe and effective radical organic syntheses devoid of dangerous by-products.

### SUMMARY OF THE INVENTION

The present invention provides an exemplary use of hypohalites, such as hypochlorous acid, sodium hypochlorite and potassium hypoiodite, as dual-radical reagents for the synthesis of pattern-specific-compounds. In this regard, unlike most synthetic methodologies that use one reagent to produce one chemical action, the method employed in the present invention targets two selective sites of a starting material using the dual-radical reagent to achieve the overall reaction. According to an embodiment, the dual-radical reagent is sequential in nature: first, the hypohalite radical removes a hydrogen atom from an appropriate donor generating hydroxyl radical as the second part of the radical system , where formation of the hydroxyl radical hinges entirely on the reaction of the hypohalite radical. As such, only those compounds containing the correct pattern in the starting material can react. Such reactions will generally be safe and produce no toxic compounds. For example, sodium chloride and water are the usual by-products.

According to an embodiment, the following chemical transformations with pattern-specific starting materials can be achieved with the exemplary process:
a. Cyclization reactions of compounds containing a phenol group and an amino group. Examples include the synthesis of the known dopaminochrome and indole.
b. Dehydrogenation reactions of compounds containing two phenol groups in the same ring, or a phenol and an amino group in the same ring. Examples include the synthesis of the known 1,2 and 1,4 benzoquinones and 1,2 and 1,4 iminoquinones.
c. Hydroxylation reactions of compounds containing a phenol group and a hydrogen on an adjacent carbon to the phenol (ortho position) or two carbons apart (para position). Examples include the synthesis of the unreported tocatechols of 6-tocopherol and the known E-quinone from α-tocopherol.
d. Decarboxylation reactions, with or without cyclizations, of starting materials containing an amino group and a carboxyl (acid) group attached to the same carbon. Examples include the synthesis of the known compounds indole, 1-pyrroline.

### BRIEF DESCRIPTION OF THE DRAWINGS

Some aspects of the disclosure are herein described, by way of example only, with reference to the accompanying drawings. With specific reference now to the drawings in detail, it is stressed that the particulars shown are by way of example and are for purposes of illustrative discussion of embodiments of the disclosure. In this regard, the description, taken with the drawings, makes apparent to those skilled in the art how aspects of the disclosure may be practiced.

FIG. 1 illustrates a decomposition of hydrogen peroxide by the enzyme catalase.

FIG. 2 illustrates a mechanism of reaction of the hydroxyl radical generator K₃HFe(CN)₆ with hydrogen peroxide, and quenching of the hydroxyl radical.

FIG. 3 illustrates the formation of a 1,4-iminoquinone by the action of potassium ferricyanide on p-aminophenol.

FIG. 4 illustrates a mechanism of reaction of an enzyme catalase on hydrogen peroxide through the formation of a hydroxyl radical intermediate.

FIG. 5 illustrates a formation of 1,4-iminoquinone from ap-aminophenol by the hydroxyl radical produced in the reaction of catalase with hydrogen peroxide.

FIG. 6 illustrates the formation of dopaminochrome from dopamine by the hydroxyl radical produced in the reaction of catalase with hydrogen peroxide.

FIG. 7 illustrates the mechanism of the formation of dopaminochrome from dopamine via the reaction of catalase with hydrogen peroxide.

FIG. 8 illustrates the reaction of hydrogen peroxide with sodium hypochlorite to produce molecular oxygen.

FIG. 9 illustrates the reaction of hydroquinone with sodium hypochlorite to produce p-benzoquinone via a dehydrogenation reaction.

FIG. 10 illustrates the mechanism of the reaction of hydroquinone with sodium hypochlorite of FIG. 9 that demonstrates the dual-radical character of hypochlorite.

FIG. 11 illustrates a mechanism of the reaction of hydrogen peroxide with sodium hypochlorite via the dual-radical character of hypochlorite.

FIG. 12 illustrates the mechanism of the reaction of 1,4-aminophenol with sodium hypochlorite to produce 1,4-iminoquinone and demonstrates the dual-radical character of hypochlorite.

FIG. 13 illustrates the mechanism of the formation of dopaminochrome by reaction of two units of sodium hypochlorite with one unit of dopamine via the dual-radical system of hypochlorite.

FIG. 14 illustrates the mechanism of the reaction of α-tocopherol with sodium hypochlorite to produce E-quinone via the dual-radical system of hypochlorite.

FIG. 15 illustrates the mechanism of the formation of the two isomers of E-catechol from δ-tocopherol (a vitamin E) with sodium hypochlorite via the dual-radical system of hypochlorite

FIG. 16 illustrates the mechanism of the formation of indole and 1-pyrroline by decarboxylation with sodium hypochlorite via the dual-radical system of hypochlorite.

FIG. 17 illustrates the reaction of potassium iodide with hydrogen peroxide to produce potassium hypoiodite and reactions of the potassium hypoiodite with hydrogen peroxide to produce molecular oxygen and with 1,4-aminophenol to produce 1,4-iminoquinone via the dual-radical system of the hypoiodite.

### DETAILED DESCRIPTION OF THE INVENTION

These descriptions are intended to illustrate some particular embodiments of the disclosure, and not to exhaustively specify all permutations, combinations and variations thereof.

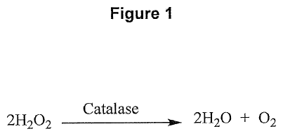

According to an embodiment, one of the most abundant radicals in the animal environment is the hydroxyl radical (OH) produced naturally by enzymes but also by the interplay of compounds such a ferric iron (Fe⁺³) and ascorbic acid (vitamin C). The hydroxyl radical produced by nature is used as a tool to perform essential reactions, it also serves as a signaling molecule of vital conditions and to destroy invading attackers such as viruses and bacteria. It was found that the enzyme catalase, present extensively in the human body to perform the conversion of hydrogen peroxide (H₂O₂) to molecular oxygen (O₂) and water (H₂O) as shown as the overall reaction in FIG. 1, generates the hydroxyl radical as an intermediate

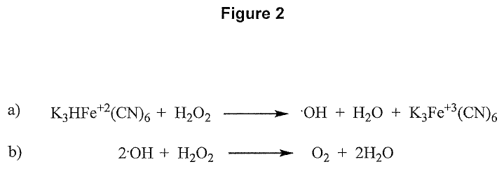

According to an embodiment, by using reported crystal structures of the enzyme and the hydroxyl radical generator K₃HFₑ(CN)₆, that also converts H₂O₂ to O₂ and H₂O but through the formation of OH radicals as shown in FIG. 2, the mechanism of action of catalase has been elucidated.

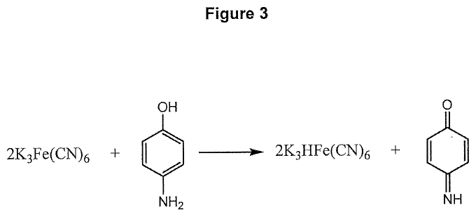

According to an embodiment, the catalase enzyme has in its native state a hydrogenated heme iron (heme-H) in a low oxidation state in close proximity to the oxygen of the phenol tyrosine that is actually a tyrosyl radical. Similar to the synthesis of K₃HFe(CN)₆ shown in FIG. 3, catalase uses the equivalent reactions of FIG. 2 and FIG. 3 to achieve similar process in a catalytic fashion.

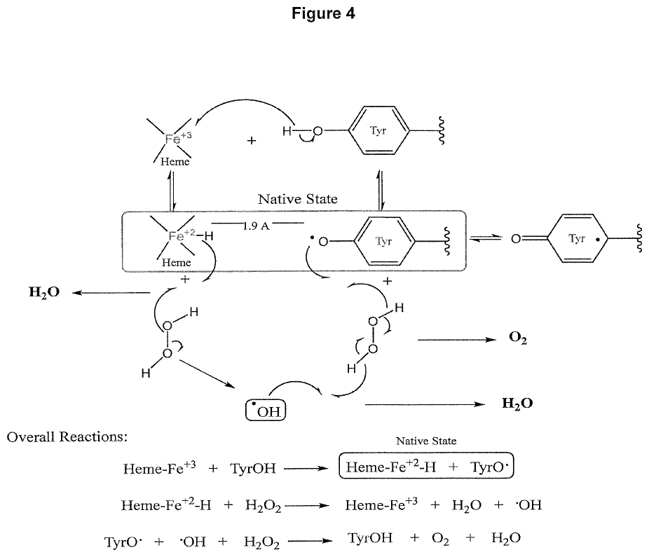

Based on the above description, the mechanism of catalase is shown in FIG. 4 where the reduced heme-H reacts with H₂O₂ to produce oxidized heme, one OH radical plus water. The tyrosyl radical participates with the OH radical to remove hydrogens from the peroxide to produce molecular oxygen and a tyrosine molecule that repeats the cycle by reacting with the oxidized heme.

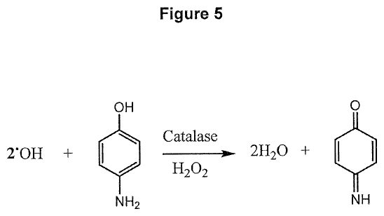

According to an embodiment, to demonstrate the production and involvement of the hydroxyl radical in the catalase mechanism, the same reaction of FIG. 3 with 1,4-aminophenol was performed producing the red 1,4-iminoquinone as shown in FIG. 5.

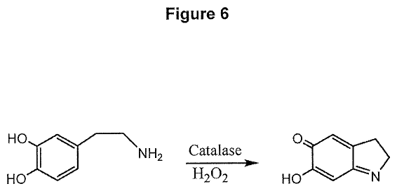

Furthermore, a reaction similar to the one displayed in FIG. 5 can be obtained using dopamine hydrochloride, a water-soluble catecholamine involved in numerous processes in the human body. When dopamine hydrochloride is added to the catalase reaction, the strongly red dopaminochrome is produced as shown in FIG. 6.

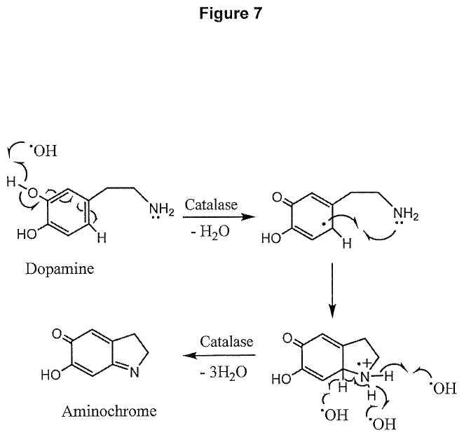

According to an embodiment, despite the fact that FIGS. 3, 5, and 6 show the involvement of an amino (NH₂) group, the hydroxyl radical is unable to attack the NH₂ hydrogens directly as demonstrated by its lack of reactivity with aliphatic and aromatic amines. The hydroxyl radical exclusively attacks phenolic hydrogens and the OH group of vitamin C, thus, the mechanism of FIGS. 3, 5, and 6 is explained by the contribution of electrons from the NH₂ group to a preformed aromatic radical as shown for dopamine in FIG. 7.

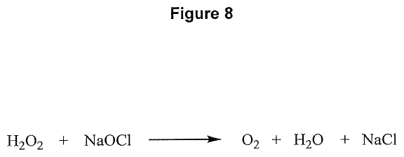

According to an embodiment, the importance of the result of FIG. 7 lies on the fact that dopaminochrome is the chemical involved in serious neurologic illnesses such as Parkinson and Alzheimer diseases, and the demonstration here of its simple formation through hydroxyl radicals has serious health issues. On that vein, other natural sources of hydroxyl radicals were sought and a forgotten published report of the radicals being formed from the reaction of sodium hypochlorite (NaOCl) with hydrogen peroxide shown in FIG. 8 was of interest because the hypochlorite ion COCl) is an abundant defense product of the immune system, formed in neutrophils, monocytes and many other cells by the ubiquitous enzyme myeloperoxidase (MPO).

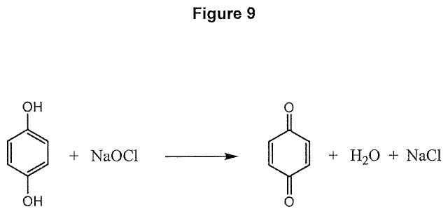

According to an embodiment, the reaction of FIG. 8 seems simple enough so the foitnation of the reported hydroxyl radicals represents a puzzle worth pursuing. With the knowledge acquired with the hydroxyl generator, we addressed the problem by reacting hypochlorite with hydroquinone, a hydrogen peroxide equivalent, as shown in FIG. 9. The reaction performed in an alcohol/water mixture is clean and fast producing the known 1,4-benzoquinone in 100% yield using a 1:1 ratio of the reagents.

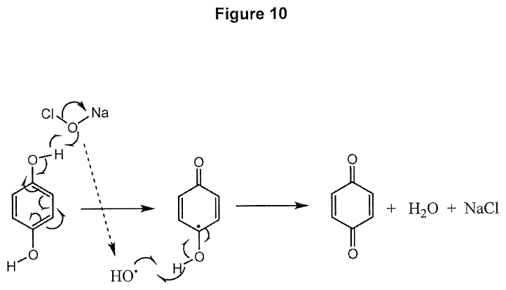

Furthermore, since the reaction of FIG. 9 proceeds equally well with two units of hydroxyl radicals, it implies that two radicals are “contained” within the NaOCl molecule and one of them should be the reported hydroxyl radical that can only be formed after removal of a hydrogen from the hydroquinone by a hypochlorite radical, as shown in FIG. 10.

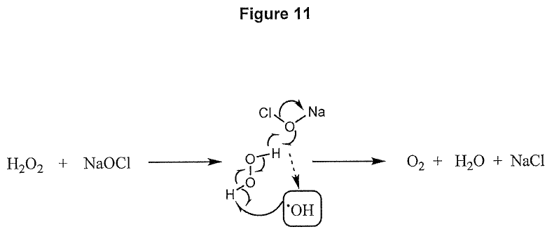

According to an embodiment, the reaction of FIG. 10 facilitates the elucidation of the mechanism of hypochlorite with hydrogen peroxide as shown in FIG. 11

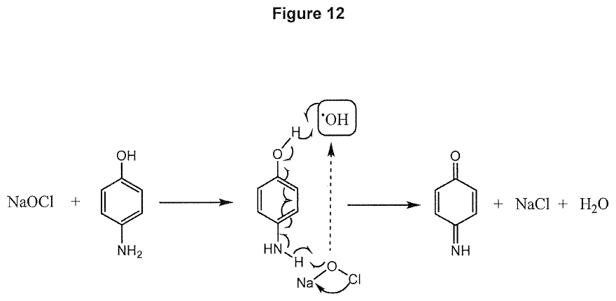

Furthermore, because the reaction is not concerted, the hydroxyl radicals can be observed with ease especially when the concentration of peroxide decreases. Application of the discovered dual radical character of hypochlorite to the reaction of 1,4-aminophenol can be done in a biphasic system of ethyl acetate/water, a method that simplifies purification of the 1,4-iminoquinone of FIG. 5. However, the mechanism here is sidelined by the strong known attraction between hypochlorite and the amino groups, which in simple situations produces chloramines, so the first step of the mechanism is the removal of the amino hydrogen by the hypochlorite radical, as shown in FIG. 12.

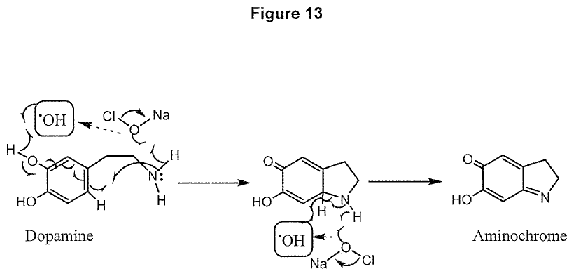

Furthermore, formation of dopaminochrome proceeds expeditiously with only two units of hypochlorite as oppose to the 4 units of hydroxyl radicals required with catalase, as shown in FIG. 13.

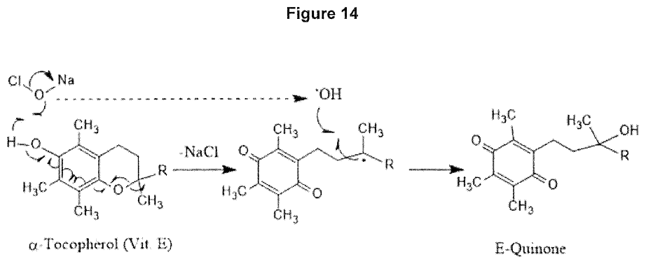

According to an embodiment, the new discovery of the dual-radical character of hypochlorite in which a hydroxyl radical is involved, ushered a search for the synthesis of compounds that are typically difficult or unattainable by other methods. In that vein, E-quinone, the metabolite of α-tocopherol, one of four vitamin E, which have been successfully produced using the hydroxyl radical was evaluated with this simple method. The reaction of α-tocopherol with one equivalent of hypochlorite in an ethanol/water mixture produces 100% yield of pure red E-quinone with sodium chloride (NaCl) as the only by-product. Following the mechanism outlined before, this synthesis is detailed in FIG. 14.

According to an embodiment, the importance of an easy and inexpensive synthesis of E-quinone lies in the fact that it allows the study of its involvement in the long known toxicity/interaction with vitamin K, a close structural analog. It has been proposed that the α-tocopherol has toxicity not shared by the β, δ and γ analogues, so the hypochlorite synthesis, a possible replication of a natural process, was used to process the δ-analogue, a compound that differs from the α-analogue in the absence of two methyl (CH₃) groups flanking the OH phenol. Since radical reactions in the aromatic ring follow the electronic rules for substitution, this reaction presents the possibility of explaining the differences in toxicity of the 4 analogues.

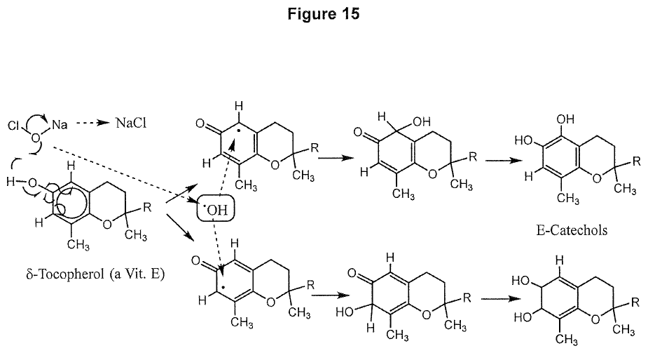

According to an embodiment, as shown in FIG. 15, attack by the hypochlorite radical can generate two tocopheryl radicals each flanking the initial OH phenol (ortho positions) in addition to the one produced with the α-analogue. When the reaction is run under the same conditions as with α-tocopherol, two compounds are produced in a 1:1 ratio corresponding to the ortho isomers, named tococatechols, with no indication of a quinone formation. This results validates the clinical conclusions that the α-analogue represents an inhibitor of vitamin K.

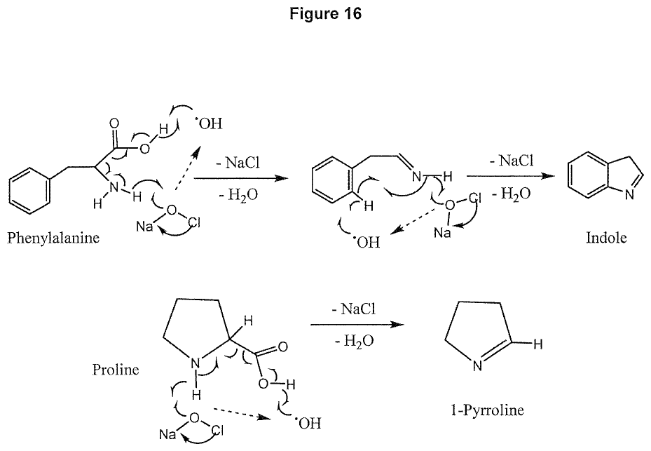

According to an embodiment, the formation of 1,4-iminoquinone and 1,4-benzoquinone represent new dehydrogenations reactions, dopaminochrome synthesis from dopamine represents a cyclization reaction, whereas E-quinone and tococatechol production are examples of new hydroxylation reactions achieved using the dual-radical character of the hypochlorite, subject of the current application. The areas of activity of this dual radical extents besides those three general methods. When phenyalanine and proline are mixed separately with hypochlorite, the known fecal metabolite indole and the semen-smelling 1-pyrroline are formed, respectively as shown in FIG. 16, in yields larger than 80%, representing yet a new method of decarboxylation with or without cyclization.

Furthermore, the simplicity of the syntheses of FIG. 16 contrasts with the existing current methods of preparation. For 1-pyrroline, for instance, one method uses 2.5 equivalents of the toxic and expensive SmI₂ to cleave the N—O bond of an oxime, and in another, SnCl₄ is employed to promote a cycloaddition between cyclopropanes and nitriles.

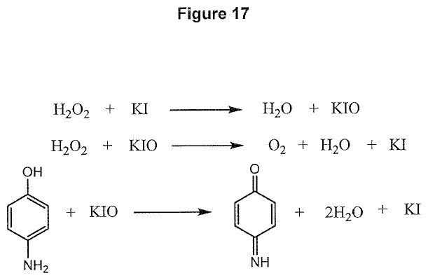

According to an embodiment, the examples given use sodium hypochlorite as the hypohalite of choice due to ample availability, simplicity of product purification and low cost, hypochlorous acid, obtained by acidification to pH 3 of sodium hypochlorite is used to produce materials that are more stable as salts such as the hydrochloride of dopaminochrome, and the hypoiodite can also be employed to perform the same reactions, but, because the hypoiodite is unstable, it is generated in the reaction vessel from the reaction between potassium iodide and hydrogen peroxide. Although hypoiodite can be used at any concentration even in catalytic amounts, the drawbacks for its use is the price of the potassium iodide compared to the hypochlorite and the price of purification of the final product due to the fact that iodine (I₂) is a by-product in this reaction, and iodine gives a yellow color to final materials. The hypoiodite is generated from the reaction of potassium iodide (KI) with hydrogen peroxide and its catalytic use stems from the fact that after reaction with the hydrogen donors, regenerates the iodide starting material, so the only consumable component is the hydrogen peroxide. As shown in FIG. 17, donors include hydrogen peroxide as well as the materials already shown for the hypochlorite.

According to an embodiment, the requirements for the dual-radical formation process makes the reactions: a) very selective, b) prevents the formation of secondary products, and c) releases non-toxic by-products, namely salt and water. Further, the reactions can be completed (with purified products) relatively quickly, making the processes very cost-effective.

According to an embodiment, four types of general reactions are covered by this dual radical method in which the basic series of events are outlined in the following general diagram using sodium hypochlorite as an example:

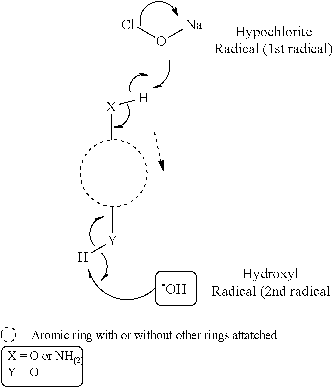

1. Cyclization reactions of compounds containing a phenol group and an amino group. Examples include the synthesis of dopaminochrome and indole.
2. Dehydrogenation reactions of compounds containing two phenol groups in the same ring, or a phenol and an amino group in the same ring. Examples include the synthesis of quinones and iminoquinones.
3. Hydroxylation reactions of compounds containing a phenol group and a hydrogen on an adjacent carbon to the phenol (ortho position) or two carbons apart (para position). In some ring systems containing a free phenol and substituted phenol in the para position, hydroxylation will occur outside the phenol ring when the substituted ring is opened up generating a radical. Examples include the syntheses of tococatechols and E-quinone.
4. Decarboxylation reactions, with or without cyclizations, of compounds containing an amino group and a carboxyl (acid) group attached to the same carbon. Examples include the synthesis of indole and 1-pyrroline.

### SYNTHETIC METHODOLOGY AND EXAMPLES

General Methods using Sodium Hypochlorite, Hypochiorous acid or Potassium Hypoiodite:

To a solution in water, or water/alcohol of the substrate (compound to undergo the reaction) is added the number of equivalents of sodium hypochlorite required to produce the reaction. The amount of equivalents of the hypochlorite are calculated from the balanced equation of the process based on the conditions of the dual radical system established above. After completion of the reaction, estimated by the disappearance of the starting material observed using thin layer chromatography (TLC), the product is isolated by extraction or by concentration followed by extraction with a solvent of appropriate polarity. If hypochlorous acid is indicated for stability of the final product, as in the synthesis of dopaminochrome, the reactions above are run on a HCL pre-acidified (pH 3) solution.

For compounds that are soluble in nonpolar solvents, a biphasic system of ethyl acetate/ water is also appropriate. As such, the substrate is dissolved in the ethyl acetate and the mixture set above the water solution of sodium hypochlorite and left undisturbed until completion (about 1-2 hours) as judged by TLC. The mixture is separated using a separatory funnel and the organic phase is passed through a short silica gel or alumina plug and concentrated.

### Cyclization Reactions

Synthesis of dopaminochrome: To a solution of dopamine in water, one equivalent of sodium hypochlorite acidified to pH 3 with HCl in water was added. After the evolution of carbon dioxide (CO₂) had subsided (about 5 minutes) dopaminochrome.HC1 was isolated by concentrating under high vacuum followed by desalting. The resulting red powder was obtained in 85% yield and the purity of the material was verified by melting point, nuclear magnetic resonance (NMR) and mass spectroscopy (MS).

Synthesis of Indole: To a solution of phenylalanine in water are added two equivalents of sodium hypochlorite in water. After the evolution of carbon dioxide (CO₂) has subsided, the indole produced is isolated by extracting it with ethyl acetate. The purity of the material was verified by melting point, nuclear magnetic resonance (NMR) and mass spectroscopy (MS). Yield is 90%.

### Dehydrogenation Reactions

Synthesis of 1,4-Benzoquinone: To a hydro/alcoholic solution of hydroquinone is added one equivalent of sodium hypochlorite in water. The mixture is stirred for 30 minutes, concentrated, and the solid produced extracted with alcohol, filtered through a plug of silica gel and concentrated to produce pure p-Quinone in 100% yield.

Synthesis of 1,4-Iminoquinone: An ethyl acetate solution of p-aminophenol is suspended in water containing one equivalent of sodium hypochlorite. After approximately 2 hours and verification of reaction completion by TLC, a red 1,4-benzoquinone imine can be isolated, passed through a plug of silica gel, and concentrated to produce 70% yield of pure material as verified by NMR and MS (mass calculated, M+H=108.0439, mass observed, M+H=108.0444).

### Hydroxylation Reactions

Synthesis of E-Quinone: In an aqueous alcoholic solution of α-tocopherol (a vitamin E), one equivalent of sodium hypochlorite in water was added. The mixture was stirred for 30 minutes and the E-quinone was extracted with ethyl acetate and concentrated to produce 100% yield of a reddish oil that gave MS of 430.2409 observed vs. MS of 430.2388 calculated. The carbon NMR in CDCl₃ shows two peaks at 187.11 and 187.50 ppm corresponding to the ketone groups formed and absent in the α-tocopherol.

Synthesis of Trolo-Quinone: Trolox and sodium hypochlorite in a 1:1 mixture in alcohol/water were stirred for 30 minutes. The yellow solution produced was concentrated, extracted with alcohol, and passed through a plug of silica gel to give, after concentration, 100% of Trolo-Quinone as a yellow solid. The carbon NMR in DMSO shows two peaks at 186.73 and 187.38 ppm corresponding to the ketone groups formed and absent in the original Trolox compound.

### Decarboxylation Reactions

Synthesis of 1-Pyrroline: A solution of proline in water was mixed with one equivalent of sodium hypochlorite in water and, after 5 minutes, the mixture was extracted with ethyl acetate, cooled to 5° C., passed through a plug of silica gel, and concentrated to produce 70% of the unstable 1-pyrroline. MS found of M+H=70.0652 vs. M+H calculated of 69.0651.

Synthesis of Indole: To a solution of phenylalanine in water, two equivalents of sodium hypochlorite in water were added. After the evolution of carbon dioxide (CO₂) had subsided, the indole produced was isolated by extracting with ethyl acetate. The purity of the indole was verified by melting point, nuclear magnetic resonance (NMR) and mass spectroscopy (MS). Yield was 90%.

Synthesis of 2,3,4,5-tetrahydropyridine: To a solution of lysine in water, two equivalents of sodium hypochlorite in water were added. After the evolution of carbon dioxide (CO₂) had subsided, the 2,3,4,5-tetrahydropyridine produced was isolated by extracted with ethyl acetate. The purity of 2,3,4,5-tetrahydropyridine was verified by melting point, nuclear magnetic resonance (NMR) and mass spectroscopy (MS). Yield was 90%.

It is to be understood that the above described embodiments are merely illustrative of numerous and varied other examples which may constitute applications of the principles of the invention. Such other embodiments may be readily devised by those skilled in the art without departing from the spirit or scope of this invention and it is our intent they be deemed within the scope of our invention.

The foregoing detailed description of the present disclosure is to be understood as being in every respect illustrative and exemplary, but not restrictive, and the scope of the present disclosure provided herein is not to be determined solely from the detailed description, but rather from the claims as interpreted according to the full breadth and scope permitted by patent laws. It is to be understood that the embodiments shown and described herein are merely illustrative of the principles addressed by the present disclosure and that various modifications may be implemented by those skilled in the art without departing from the scope and spirit of the present disclosure. Those skilled in the art may implement various other feature combinations without departing from the scope and spirit of the present disclosure. The various functional modules shown are for illustrative purposes only, and may be combined, rearranged and/or otherwise modified.

What is claimed is:

1. Compounds produced using the general scheme of Formula I: 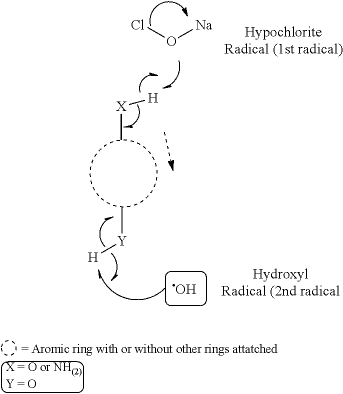
Wherein X represents an oxygen or a nitrogen atom that carries a reactive hydrogen that is removed by the hypochlorite radical to generate a hydroxyl radical that attacks another reactive hydrogen carried by Y that represents an oxygen atom. The atoms represented by X and Y may be attached directly to the aromatic ring or interspaced by other groups such a carboxyl group. Those attachments will dictate the outcome of the reaction as a cyclization, dehydrogenation, hydroxylation, decarboxylation, or a combination of those processes.

2. The chemical product of claim 1, wherein the compound is the phenylethylamine core (the core of dopamine), where two dehydrogenations take place on the hydrogens attached to the nitrogen atom and one dehydrogenation takes place at the oxygen atom, and a cyclization takes place to produce aminochromes of Formula II, where R₁, R₂ and R₃ are alkyl, alkoxy groups:

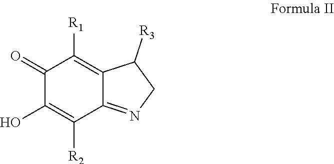

3. The chemical product of claim 1, wherein a core hydroquinone compound undergoes two sequential dehydrogenation reactions of the hydrogens attached to X═O and Y═O to produce 1,4-benzoquinones of Formula III, where R₁, R₂, R₃ and R₄ are alkyl or alkoxy groups:

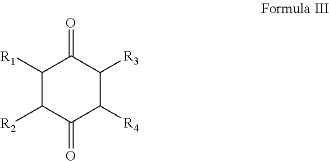

4. The chemical product of claim 1, wherein the core compound 4-aminophenol undergoes two sequential dehydrogenation reactions of the hydrogens attached to X═N and Y═O to produce 1,4-iminoquinones of Formula IV, where R₁, R₂, R₃ and R₄ are alkyl or alkoxy groups:

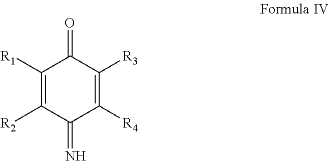

5. The chemical product of claim 1, wherein the core compound 2-aminophenol undergoes two sequential dehydrogenation reactions of the hydrogens attached to X═N and y═O to produce 1,2-iminoquinones of Formula V, where R₁, R₂, R₃ and R₄ are alkyl or alkoxy groups:

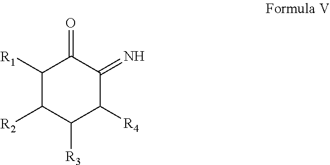

6. The chemical product of claim 1, wherein the 5, 7, 8-trimethyl-6-chromanol core (the core of α-tocopherol) undergoes a dehydrogenation reaction of the hydrogen attached to X═O followed by a hydroxylation on a generated radical to produce E-quinones of Formula VI, where R is an alkyl, carbonyl or carboxy group:

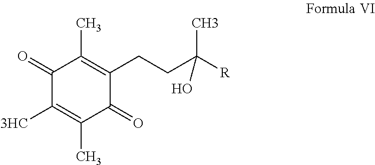

7. The chemical product of claim 1, wherein the 8-methyl-6-chromanol core (the core of δ-tocopherol) undergoes a dehydrogenation reaction of the hydrogen attached to X═O followed by a hydroxylation on the adjacent carbon to produce catechols of Formula VII, where R is an alkyl, carbonyl or carboxy group:

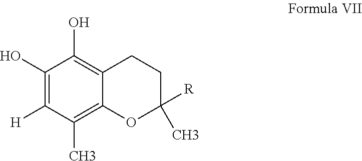

8. The chemical product of claim 1, wherein the 8-methyl-6-chromanol core (the core of δ-tocopherol) undergoes a dehydrogenation reaction of the hydrogen attached to X═O followed by a hydroxylation on the adjacent carbon to produce catechols of Formula VIII, where R is an alkyl, carbonyl or carboxy group:

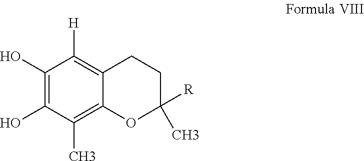

9. The chemical product of claim 1, wherein the 7, 8-dimethyl-6-chromanol core (the core of β-tocopherol) undergoes a dehydrogenation reaction of the hydrogen attached to X═O followed by a hydroxylation on the adjacent carbon to produce catechols of Formula IX, where R is an alkyl, carbonyl or carboxy group:

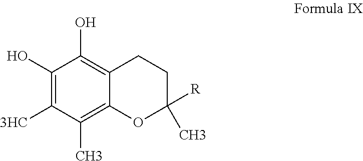

10. The chemical product of claim 1, wherein the 5, 8-dimethyl-6-chromanol core (the core of γ-tocopherol) undergoes a dehydrogenation reaction of the hydrogen attached to X═O followed by a hydroxylation on the adjacent carbon to produce catechols of Formula X, where R is an alkyl, carbonyl or carboxy group:

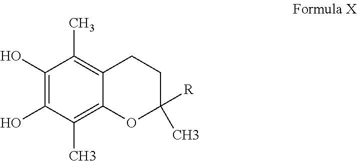

11. The chemical product of claim 1, wherein the pyrrole core (the core of proline) undergoes two sequential dehydrogenation reactions of the hydrogens attached to X═N and y═O and a decarboxylation to produce 1-pyrolines of Formula XI, where R1 and R2 are an alkyl groups:

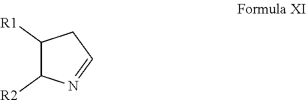

12. The chemical product of claim 1, wherein the compound core of phenyalanine undergoes two sequential dehydrogenation reactions of the hydrogens attached to X═N and y═O followed by decarboxylation and cyclization to produce indoles of Formula XII, where R₁, R₂, R₃ and R₄ are alkyl or alkoxy groups:

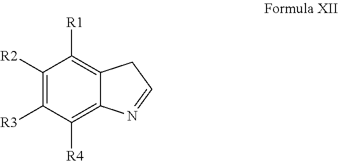

13. The chemical product of claim 1, wherein the compound core of lysine undergoes two sequential dehydrogenation reactions of the hydrogens attached to X═N and y═O followed by decarboxylation and cyclization to produce 2,3,4,5-tetrahydropyridines of Formula XIII, where R₁, R₂, R₃ and R₄ are alkyl or alkoxy groups: [chemical structure]
